(19) United States
(12) Patent Application Publication
(10) Pub. No.: US 2014/0180007 A1
(43) Pub. Date: Jun. 26, 2014

(54) SOFT ENCLOSING MEMBRANE FOR CAMERA

(71) Applicants: Avram Allan Edidin, Portola Valley, CA (US); Xiaolong Ouyang, Palo Alto, CA (US)

(72) Inventors: Avram Allan Edidin, Portola Valley, CA (US); Xiaolong Ouyang, Palo Alto, CA (US)

(21) Appl. No.: 14/136,799

(22) Filed: Dec. 20, 2013

Related U.S. Application Data
(60) Provisional application No. 61/740,672, filed on Dec. 21, 2012.

(51) Int. Cl. A61B 1/00; A61B 1/05

(52) U.S. Cl. CPC A61B 1/00142; A61B 1/05
USPC 600/122

(57) ABSTRACT

An imaging device can include: an imaging module; a lens associated with the imaging module; and an optically transmissive inflatable member surrounding the lens. The inflation tube can be fluidly coupled with the inflatable member. An inflation port can be fluidly coupling the inflation tube and inflatable member. A fastener that fastens the inflatable member can be coupled to a housing of the imaging module. A flushing tube can be fluidly coupled to a flushing port that is directed outward from a housing of the imaging module. The inflation tube and/or flushing tube are coupled to a pneumatic system that can pump fluid therethrough. The inflatable member can be an inflatable membrane, balloon, bladder, or bag.

### CROSS-REFERENCE

The present application claims the benefit of U.S. Provisional Application No. 61/740,672 filed Dec. 21, 2012, which provisional application is incorporated herein by specific reference in its entirety.

### BACKGROUND

Imaging with a camera can be performed such that the lens is subjected to unfavorable environmental conditions that impair image quality. For example, imaging with an endoscope within a body cavity or tissue space can be subject unfavorable environmental conditions, such as debris, blood, body fluids, and the like, that cause the image to lack clarity with respect to the image target. The target of the image can be clouded by the environmental conditions so that it is difficult to perform a task associated with the image, such as a surgery. Saline or other proper solutions may be infused into an imaging area in order to reduce murkiness; however, the body fluid components and debris can still impair the image quality. Thus, there is a need in the art for an improved device that increases image quality by imparting favorable environmental conditions to an imaging area.

### SUMMARY

In one embodiment, an imaging device can include: an imaging module; a lens associated with the imaging module; and an optically transparent inflatable member surrounding the lens. In one aspect, an inflation tube can be fluidly coupled with the inflatable member. In one aspect, an inflation port can be fluidly coupling the inflation tube and inflatable member. In one aspect, a fastener can fasten the inflatable member to a housing of the imaging module.

In one embodiment, a flushing tube can be fluidly coupled to a flushing port that is directed outward from a housing of the imaging module. In one aspect, the inflation tube and/or flushing tube are coupled to a pneumatic system that can pump fluid therethrough.

In one aspect, the inflatable member is an inflatable membrane. In one aspect, the inflatable member is a balloon. In one aspect, the inflatable member is a bladder. In one aspect, the inflatable member is a bag. In one aspect, the inflatable member is prepared from a polymer that is transparent. In one aspect, the inflatable member is prepared from an elastomer that is transparent. In one aspect, the inflatable member is prepared from a latex that is transparent. In one aspect, the inflatable member is prepared from polyurethane, nylon, polyethylene, polypropylene, or the like.

In one embodiment, the inflatable member is on a distal end of a shaft having the imaging module at the distal end. The shaft can be an endoscope.

In one embodiment, an imaging method can include: providing an imaging device having a housing containing an imaging unit with a lens and an inflatable member coupled with the housing with the lens contained within the inflatable member; and imaging an imaging area through the inflatable member. In one aspect, the method can include inflating the inflatable member. In one aspect, the method can include deploying the device to the imaging area to be imaged with the inflatable member collapsed. In one aspect, the method can include selectively inflating the inflatable member so that the inflatable member is a desired distance from the lens. In one aspect, the method can include deflating the inflated inflatable member. In one aspect, the method can include pressing the inflatable member against the imaging area. In one aspect, the method can include infusing a space adjacent to the imaging area with an infusion solution around the inflatable member.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIGS. 1A-1C illustrate an embodiment of an imaging device having an inflatable balloon member coupled to a distal end of the elongate member so as to cover the imaging components.

FIGS. 2A-2B illustrate an embodiment of an inflatable cover for an imaging device.

FIGS. 3A-3B illustrate an embodiment of an inflatable bladder for an imaging device.

FIG. 4 illustrates an embodiment of an imaging device having a base that can coupled to the elongate imaging member with the inflatable member on the distal end of the elongate imaging member.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention is related to imaging devices that include a transparent member around a lens of the imaging device. That is, the transparent member provides a transparent compartment around the lens of the imaging device such that the imaging device can take an image through the transparent member in order to image subject matter outside of the transparent compartment. The transparent member can be spaced away from the lens so that there is a gap therebetween. Accordingly, the transparent member allows for capturing images with a camera in manner that can be performed such that the lens is not subjected to unfavorable environmental conditions that impair image quality. The transparent member provides a transparent compartment that contains the lens so that the transparent member is subjected to the unfavorable environmental conditions such that the lens is retained in favorable conditions within the transparent compartment. The transparent compartment is subjected to the unfavorable environmental conditions so that the lens is kept clean and capable of taking quality images in such an unfavorable environment because the images can be obtained through the transparent member. For example, the imaging device having the transparent member forming the transparent compartment around the lens can allow for imaging within a body cavity or tissue space that can include unfavorable environmental conditions, such as debris, blood, body fluids, and the like, that can cause ordinary endoscope images to lack clarity with respect to the image target, where the invention with the transparent member provides for high quality images in such an environment. The target of the image can be clouded by the environmental conditions so that it is difficult to perform a task associated with the image, such as a surgery; however, the transparent member forming the transparent compartment over the lens can allow for the transparent member to be pressed against the target so that a clear image can be obtained by the imaging device. The transparent compartment can be kept clear so that the transparent member can push away the clouded environmental conditions and allow a clear image to be obtained. Saline or other proper solutions may be infused into the transparent compartment to maintain a clear imaging volume. Also, such saline or other proper solutions may be infused into the imaging area in order to reduce murkiness; however, the body fluid components and debris can still impair the image quality, which is overcome by the transparent member pushing such body fluid components and debris aside and away from the image target. Thus, the present invention of an imaging device with a transparent member forming a transparent compartment around the lens of the imaging device provides an improved device that increases image quality by imparting favorable environmental conditions to an imaging area. In one aspect, the transparent member can be a membrane. In one aspect, the transparent member can form the transparent compartment as a transparent bag. In one aspect, the transparent compartment has a fixed volume. In one aspect, the transparent compartment has a variable volume that can be expanded and/or contracted as desired. In one aspect, the transparent compartment has a fixed shape. In one aspect, the transparent compartment has a variable shape that can be deformed and manipulated.

In one embodiment, the transparent member may not be fully transparent. As such it may be partially transparent, such as 90%, 80%, 70%, 60%, 50%, 40%, 30% or 25%. The transparency may be any transparency that allows for imaging through the member. This may include the member being a optically transmissive member. The member may also be colored so that the image is colored, such as red, orange, yellow, green, blue, indigo, violet, or any other coloring. Also, the image obtained can be processed, recolored, color corrected, or other processing to get a viewable image.

In one embodiment, the present invention includes an imaging device with a membrane associated with the lens such that the lens images through the membrane. The membrane can be the transparent member. The membrane can be elastomeric or otherwise stretchy or expandable (e.g., balloon), which can allow for the membrane to be in an unexpanded state associated with the lens where the membrane contacts or is adjacent to the lens and then change to an expanded sate where the lens expands outwardly and away from the lens so as to create a void. The membrane may not be stretch or elastomeric, but instead may be configured as a bag or bladder that inflates without expansion. Alternatively, the membrane can be configured as a balloon.

The imaging device can be any device that can take still images, sequential images, and/or video. CMOS imaging cameras have become widely available and many are available as well in small form factors, which allows for use in endoscope applications. The device can be configured with a reduced size (e.g., unexpanded) and used in confined spaces, such as 0.5 to 2 mm in a tissue space, and then the transparent member can be expanded to expand the imaging area.

The transparent member can be prepared of an elastomeric material that is translucent, radiolucent, transparent, clear, or otherwise capable of being imaged through. That is, the imaging device captures the image through the transparent member, either when unexpanded or expanded. The transparent member can be an elastomer or balloon material that can provide for stable packaging and robustness. Furthermore, a rigid system may be unpleasant for the end-use in applications such as endoscopic imaging due to tissue sensitivity, and the transparent member can be inflated to reduce the end rigidity and provide comfort to the subject. The transparent member can be a transparent polymer membrane or sheet. The transparent member can be stretchable and expandable, or have a fixed shape when inflated so as to not expand.

While the present invention is described in connection to an endoscope, the principles may be applied to any type of imaging device. For example, the imaging system can be configured to be used in water (e.g., hydraulic applications), soil (e.g., environmental sampling), sand (e.g., fluidized support imaging), vapor (e.g., water vapor conditions), smoke, gas, air, gel (e.g., food analytic applications), or the like. The imaging system can be adapted to image internal organs or other tissues of a subject, such as a human. The imaging system may also be used to image internal areas of complex mechanical systems, and the transparent member can be used to move lubricants or other fluids away from the target of the image.

The imaging device of the present invention may be configured as a disposable imaging unit that couples to a reusable base unit. The disposable imaging unit may have one or more mechanical elements and one or more electrical elements unified into a single assembly. For instance, the disposable imaging unit can include a disposable imaging module constructed so as to permit distal articulation of a camera and light source as well as distal inflation or expansion of the transparent member. As such, the disposable imaging unit may include both mechanical elements and electrical elements. A disposable imaging unit may be advantageous in medical uses, such as being configured as an endoscope.

In these and other embodiments, power to the camera and light source and mechanical force(s) and fluid pressure may be coupled across an interface between the disposable imaging unit and a corresponding non-disposable or reusable base unit. The ability to transfer power and mechanical forces and fluid pressure allows for the disposable imaging unit to have the functionality of imaging and mechanical manipulation and fluid inflation of the transparent member once the disposable imaging unit is coupled to the reusable base unit. As such, the interface can provide an electronic connection system that includes a first electronic connection interface on the disposable imaging unit side and a second electronic connection interface on the reusable base unit side. The electronic connection system allows for electronic signals as well as power to be passed from the reusable base unit to the disposable imaging unit and vice versa. The interface can also include a mechanical connection system that includes a first mechanical connection interface on the disposable imaging unit side and a second mechanical connection interface on the reusable base unit side, which first and second mechanical connection interfaces connect in order for mechanical forces, tension, compression, or other bias to be transferred from the reusable base unit side to the disposable imaging unit. The disposable imaging unit can then perform mechanical manipulations via forces generated and/or propagated by the reusable base unit. The mechanical connection system facilitates transfer of forces, tension, compression, or other bias or mechanical manipulation to the disposable imaging unit for mechanical functionality. The interface can also include a fluid pressure connection system that includes a first fluid tube and mechanical connection and a second fluid tube and a mechanical connection that can be coupled together to fluidly couple the first fluid tube with the second fluid tube so that fluid can be pressurized therein to inflate the transparent member. The transparent member can be selectively inflated and deflated with proper fluid, such as air or saline, etc.

The reusable base unit can be any standard medical base unit that has electronic and mechanical modules to provide electronic data and mechanical manipulation to the imaging unit. The imaging unit can include a pressure system to provide fluid pressure to the fluid tubes to inflate the transparent member. The imaging unit can include a support member, mechanical actuator, electrically conductive elements, fluid pressure actuator, one or more imaging modules, one or more mechanical components, and an electronic/mechanical/fluid interface. The support member can be any type of support member common medical devices that be used percutaneously or within some body cavity or lumen, such as a catheter-type support member. The mechanical actuator can be located in the support member and can extend from a distal end to a proximal end of the support member. The electrically conductive elements can be located within the support member and configured for providing electrical power to one or more electrical elements, such as imaging and/or lighting modules located in the support member. The imaging modules can be configured to record images as well as illuminate the subject to be recorded. The mechanical components can be configured to articulate the end of the support member and/or provide some mechanical function that may or may not be therapeutic or medical in nature. The electronic/mechanical interface can be at the proximal end of the support member, and can be configured to receive data, electrical power, and mechanical forces from the reusable base unit. The electronic/mechanical interface can also be configured to be removably coupled with the reusable base unit through a corresponding and compatible interface on the reusable base unit. The fluid pressure system can either generate proper fluid pressure itself or obtain the fluid pressure from an outside pressure source. The fluid pressures system can transfer the fluid pressure to inflate the transparent member or reduce the fluid pressure to deflate the transparent member.

The electronic/mechanical/pressure interface can include a force transfer contact member and a plurality of electrical contacts and one or more fluid tube connectors. The force transfer contact member can be coupled to the mechanical actuator and configured to transfer mechanical forces from the base unit to the mechanical actuator. That is, mechanical forces on the reusable base unit side are transferred to the mechanical actuator across the force transfer contact member. The plurality of electrical contacts can be coupled to the electrically conductive elements in the support member and configured to transfer electrical power and electronic data from the base unit to the plurality of electrically conductive elements. One or more of the plurality of electrical contacts can be configured to transfer electronic data from the disposable unit to the base unit. The fluid tube connectors can be fluidly coupled to the transparent member and to the pressure system.

The force transfer contact member can be configured in various ways in order for a force to transferred thereacross such that the mechanical actuator can be actuated from the base unit. The force transfer contact member can include any type of fastener that fastens the mechanical actuator to a mechanical controller in the base unit. The fastener can be any type of that allows coupling with force to be transferred thereof, which can include a nut and bolt, hook and eyelet, snap coupling, magnet or adhesive. In one example, the force transfer contact member can include a permanent magnet member, ferromagnetic member, or electromagnet member with the base unit side having a corresponding force transfer contact member. When the force transfer contact member includes an adhesive, any adhesive material suitable for the materials of the actuator member and other components can be used, where cyanoacrylates, epoxies, silicones, or the like can be used.

The electronic/mechanical/pressure interface can include one or more engagement features that are configured to removably engage corresponding engagement features of the base unit. The engagement features of the base unit and disposable imaging unit, when coupled together, prevent inadvertent decoupling of the disposable imaging unit from the base unit.

The disposable imaging unit can have various configurations, such as those illustrated and described herein, were features of one figure and description can be combined with any other features of any other figure or embodiment.

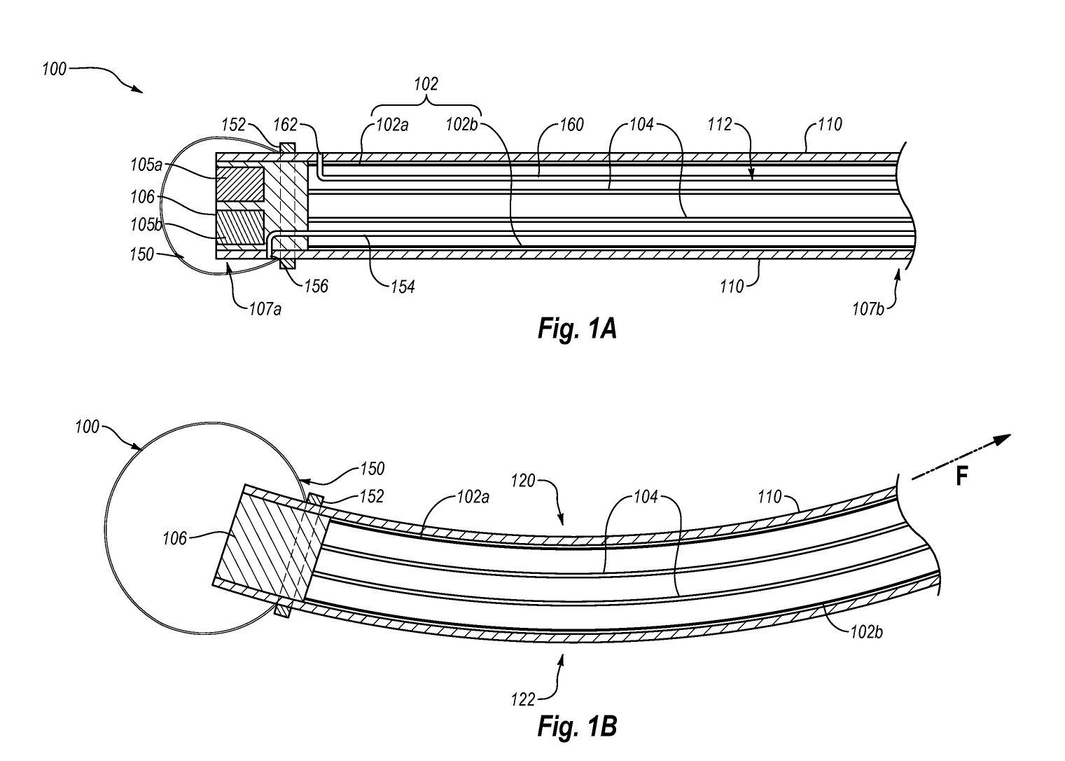

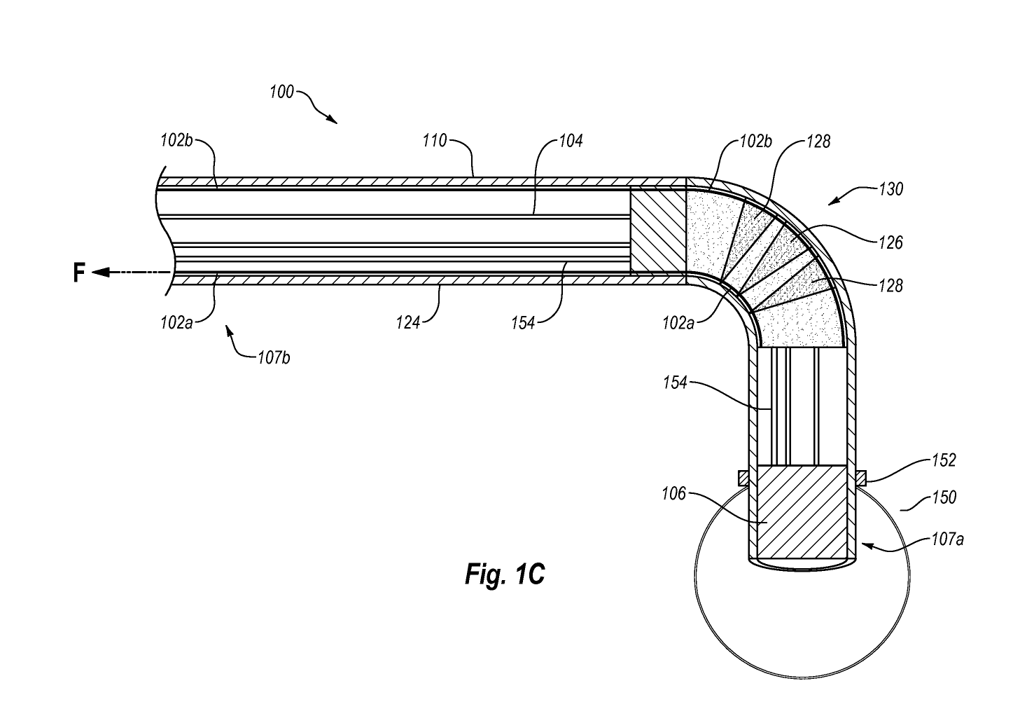

FIG. 1A illustrates an imaging unit 100 including an inflatable member 150 at the distal end 107a. The imaging unit 100 can include a fastener 152 that fastens the inflatable member 150 at the distal end. The inflatable member 150 can be coupled to an inflation pathway 154 (e.g., fluid pathway, air, saline, etc.), such as a tube that can be used to inflate the inflatable member 150. The inflation pathway 154 opens into the inflatable member 150 at an inflation port 156. The inflation port 156 is between the inflatable member 150 and the fastener 152 so that fluid from the inflation pathway 154 inflates the inflatable member 150. The inflation of the inflatable member 150 can be via fluid or gas or air. The inflatable member 150 is clear or transparent so that the imaging unit 100 can image through the inflatable member 150. For example, the inflatable member 150 can be pressed adjacent to the tissue to be imaged such that the inflatable member 150 pushes any unfavorable environment away for better imaging.

Additionally, a fluid pathway 160 can also be included that is connected to a port 162 such that the port 162 is directed adjacent but outside of the inflatable member 150. As such, the fluid expelled from the port 162 can be used to flush or clean the imaging area. Any clear fluid, such as water, saline, air, nitrogen, or the like can be used to clean the imaging area.

The imaging unit can include both mechanical elements 102 for maneuvering the distal end during deployment and electrical elements 104 for operating the imaging sensor, such as a camera module 105a. The mechanical elements 102 can include one or more mechanical actuators. The disposable imaging unit 100 can include a disposable imaging module 106 that has a camera module 105a and light source 105b. The imaging module 106 is located at a distal end 107a of the imaging unit that is opposite of a proximal end 107b as in the illustrated embodiment of FIG. 1A. As depicted in FIG. 1A, the mechanical elements 102 include two thin wires 102A, 102B or other mechanical actuators running along a support member 110 of the imaging unit 100. Here, the support member 110 includes a tubular body having an internal lumen 112 that contains the mechanical elements 102 and electrical elements 104 and fluid pathway 154 and fluid pathway 160.

The support member 110 may be made of a flexible material, for instance. Such a configuration may enable articulation of the imaging unit 100 to bend in one or more planes. In particular, by applying sufficient tension in an appropriate direction to one of the mechanical elements 102 (e.g., wire 102a), a length of the mechanical element 102a can be drawn to the base unit so as to effectively shorten one side 120 of the support member 110 compared to the other side 122 so as to cause a curvature to be created in the support member, as generally illustrated in FIG. 1B. The arrow shows the mechanical element 102a being pulled proximally or toward the base unit.

In more detail, FIG. 1C depicts a disposable unit 100 having a support member 110 including a sheath 124 and a bending section 126 that bends when tension (shown by arrow) is applied to one of the mechanical elements 102a. The bending section 126 can be uniform or segmented with segments 128 as shown. The bending section 126 can have a bendable sleeve 130 covering the segments 128. The bending section 126 allows for the distal end 107a to bend relative to the proximal end 107b. The bending section 126 can be configured to bend in the two planes or in any direction by articulation of one or more mechanical elements 102. Two mechanical elements 102 can cooperate to bend the distal end 107a in the plane of the figure page as shown. Two other or orthogonally oriented mechanical elements 102 can cooperate to bend the distal end 107a in the plane that bisects or is normal to the plane of the figure page. The use of two mechanical elements 102 for each bending plane allows for bending in both directions, such as the direction shown which appears down, an upward direction opposite as shown, or into or out from the page as well as in any direction of bending therebetween in a combination of the planes. The fluid pathway 154 is also bendable. The bending allows the inflatable member 150 to be directed toward a tissue of desire, such as by 90 degrees.

In these and other embodiments, an electrical interface (not shown) may be provided to transfer electrical power to the camera module 105a and light source 105b in the imaging unit 100 from a corresponding base unit in any suitable manner. The electrical interface can be any specific electrical interface or any suitable type of electrical interface now known or later developed.

Additionally, a mechanical transfer interface (not shown) may be provided that permits the imaging unit 100 to be removably attached to a corresponding base unit, and transfers force(s) between the base unit and the imaging unit 100 for operating the mechanical elements 102 of the imaging unit 100. Accordingly, some embodiments disclosed herein provide a connectable-detachable interface that transfers both mechanical force and electrical power through the same connector.

Additionally, a pressure, pneumatic or other fluid flow interface can be provided that permits fluid, such as liquid, gas, or air, to be selectively flowed through the fluid pathways 154, 160 in the shaft in order to selectively inflate the inflatable member 150 or to exit the port 162 to flush the imaging environment. Any system that can force a fluid through for inflation or flushing can be used.

The shaft of the imaging device can include one or more lights 105b. A plurality of lights 105b can be positioned around distal end 107a of shaft, such as around the lens. The lights 105b can be arranged as annular light on annular distal end of the shaft. Each of the lights can have one or multiple LED chips.

In one option, the imaging device 100 can have devoid of a lighting module. Here, external lighting devices can be used.

In one embodiment, the present invention can include an imaging device having one or more imaging modules.

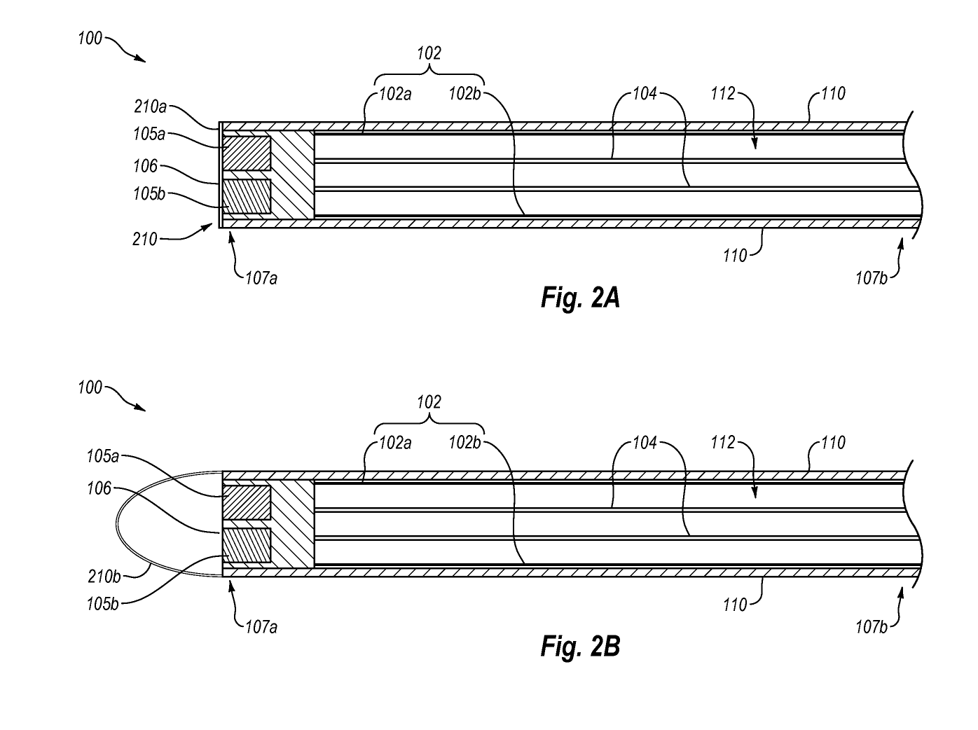

FIGS. 2A-2B illustrate another embodiment, where the transparent member 210 is allowed to lay flat against the distal end 107a when in a deflated condition 210a, and then expand away from the distal end 107a when in an inflated condition 210b. The transparent member 210 is configured as a balloon against the distal end 107a and allowed to deflate for placement and inflate for imaging. Here, the transparent member 210 can be molded and formed with the device so a fastener is not needed, but a fastener can be used to mount the transparent member 210

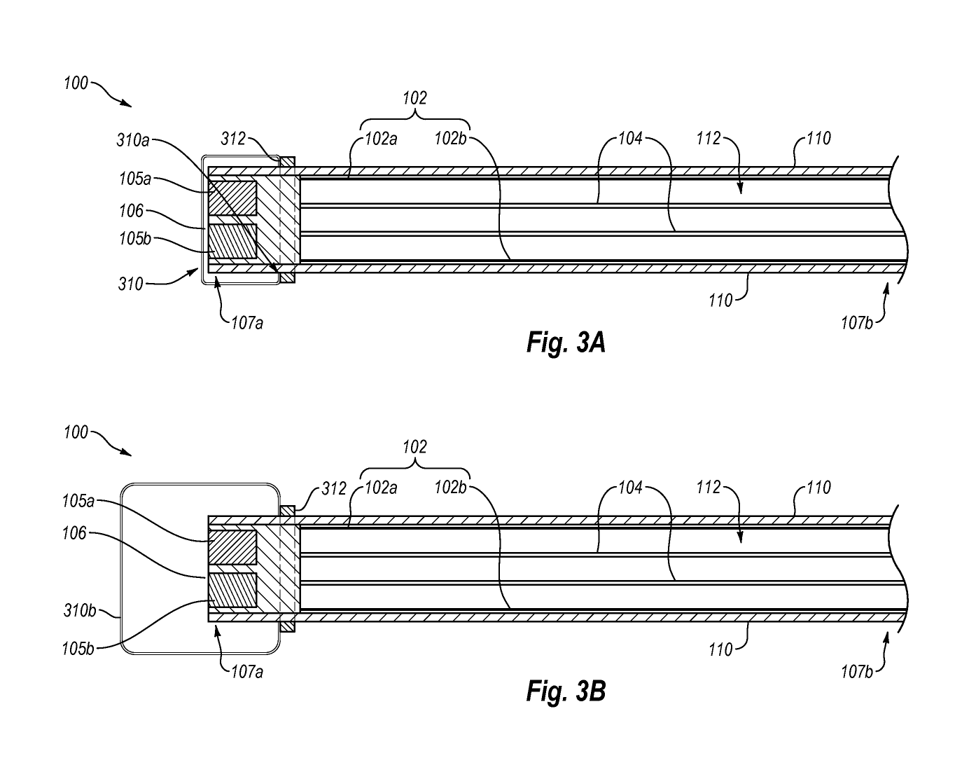

FIGS. 3A-3B illustrate another embodiment, where the transparent member 310 is a bladder held to the device with a fastener 312. However, a fastener 312 may not be needed if the transparent member 310 if adhered or integrated with the device. As shown, the transparent member 310 is a bladder with a shape (e.g., rectangle) that can be deflated (FIG. 3A) into a deflated condition 310a, and then inflated into an inflated condition 310b.

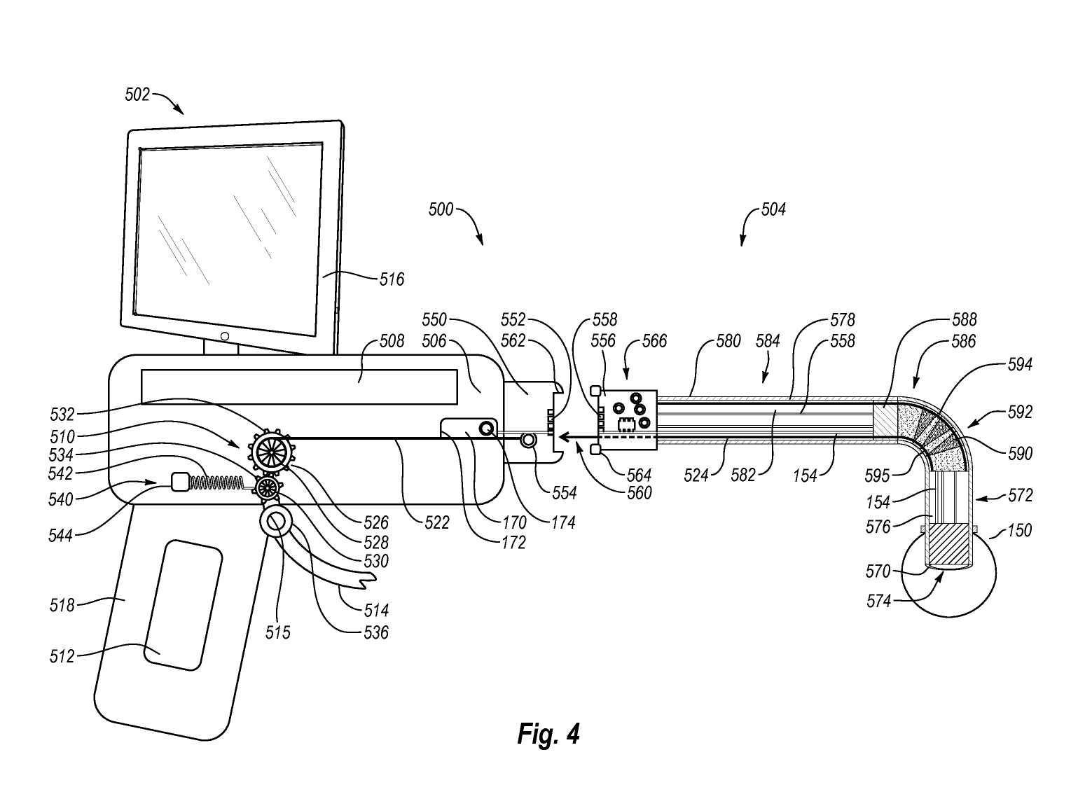

FIG. 4 illustrates an imaging medical device 500. The medical device 500 can be configured with the components to function as a cytoscope, however, other components can be included for other functionalities. In the cytoscope configuration, the medical device 500 can include imaging modules. The medical device 500 can include a reusable base unit 502 and a disposable imaging unit 504. The base unit 502 can include a main housing 506 that contains a system control module 508 which can control one or more electronic components of the base unit 502 and/or imaging unit 504. The main housing 506 can also include a mechanical system 510 that is coupled to a mechanical activating member 514 (e.g., activating lever) and mechanical deactivating member 515 (e.g., activating button). The main housing 506 may also include a power supply 512, which can be portable (e.g., batteries) or coupled with a wall power supply (e.g., cord to be plugged into wall outlet). The main housing 506 can include a gun shape with a handle 518 as illustrated or it may have some other shape configuration. The main housing 506 can also include a display screen 516 that can be integrated or couplable therewith. In one option, the disposable imaging unit 504 may be configured to be reusable and may have a disposable sheath (not shown). The main housing 506 can include a pneumatic system 172 that can provide pressurized fluid to the inflatable member 150. The pneumatic system 172 can include any type of system that can create pressurized fluid. The pneumatic system 172 can include a pressure switch 174 that allows for selectively increasing or decreasing pressure in the pneumatic lines 170, 154 to the inflatable member 150. The pneumatic line 170 is included in the main housing 506 and can fluidly couple with the pneumatic line 154 of the imaging unit 504 that is coupled to the inflatable member 150. Thus, activating the pneumatic system 172 with the pressure switch 174 can pressurize fluid in the pneumatic line 170 to pressurize the pneumatic line 154 and pressurize and inflate the inflatable member 150. The pneumatic line 170 can include a male or female fastener to be received with a corresponding fastener of the pneumatic line 154 that can be coupled together when the imaging unit 504 is coupled to the base unit 502.

The main housing 506 can include any type of mechanical system 510 that can transfer a force to a mechanical actuating member in order to bend the distal end of the disposable imaging unit 504. The mechanical system 510 functions to bend the distal end of the disposable imaging unit 504 in one or more planes, in either direction. As illustrated, one or more main force cables 522 can be included in the main housing 506 that can be functionally coupled to articulating force cables 524 of the disposable imaging unit 504, where activation of the articulating force cables 524 moves or bends the distal end of the disposable imaging unit 504.

The mechanical system 510 is illustrated to include a geared spindle 526, where one geared spindle 526 can be provided for each main force cable 522. In FIG. 4, only one force cable 522 and geared spindle 526 are shown; however, there could be two per bending plane in embodiments capable of bending in both directions. A total of four force cables 522 and geared spindles 526 can allow for bending in both directions in both planes an in various bending orientations therebetween by using one or more of the geared spindles to apply tension to separate force cables. The geared spindle 526 can be activated by operating the mechanical activating member 514 in order to cause the geared spindle 526 to reel the force cable 522 so as to be wound around the geared spindle 526. The wound force cable 522 is represented by spooled force cable 528. The mechanical deactivating member 515 can be operated in order to release or let out the force cable 522 in order to reduce or remove tension and let the force cable relax so that the bendable section relaxes and straightens.

The main housing 506 can also include a main interface 550, which can be configured as described herein. The main interface 550 can have base electrical connectors 552 and one or more base mechanical connectors 554, which are on the base unit 502 side. The electrical connectors 552 can be operably coupled to the system control module 508, and the mechanical connectors 554 can be operably coupled to the mechanical system 510. The disposable imaging unit 504 can include an imaging interface 556 that has imaging electrical connectors 558 and one or more imaging mechanical connectors 560. The imaging electrical connectors 558 can correspond and electronically couple with the base electrical connectors 552. The imaging mechanical connectors 560 can be of the same number as and mechanically couple with the base mechanical connectors 554. The main interface 550 can have main engagement features 562 that mate with and receive imaging engagement features 564 of the imaging interface 556.

The imaging unit 504 can include a printed circuit board 566 (PCB 566) with some imaging components or other control modules. The PCB 566 can be configured to be one time programmable (OTP) as described in U.S. patent application Ser. No. 13/094,415, which is incorporated herein by specific reference in its entirety. The PCB 566 can be adjacent to the imaging interface 556, or distally associated with the imaging interface 556.

The PCB can be electronically coupled with electronic lines 568, which can be configured as electronic wires or other electronic paths capable of transmitting power and/or electronic data. The electronic lines 568 can extend along the length of the imaging unit 504 to a camera module 570 located at the distal end 572. The camera module 570 can be oriented to capture images from a distal opening 574 of the imaging unit 504. Optionally, the distal end 572 can include or be configured as a camera module housing 576 that houses the cameral module 570 and has the inflatable member 150. The cameral module housing 576 can be of any suitable size; however, examples include having a length of about 2 to 20 mm, from 4 to 15 mm, or from 5 to 10 mm, and a width or diameter of about 2 mm to about 8 mm, from about 2.5 mm to about 5 mm, or from about 2.8 or 3 mm to about 4 mm.

The imaging interface 556 may also include the pneumatic couplings and can be coupled with or otherwise associated with an elongate body 578 that is positioned between the imaging interface 556 and the distal end 572. The elongate body 578 can be similar to the support member described herein, and can be tubular in shape with one or more internal lumens 582, such as lumens for the articulating force cables 524 and/or for the electronic lines 558 and the pneumatic line 154. The elongate body 578 can include a sheath 580, which can be individually disposable or the entire imaging unit 504 can be disposable. The sheath 580 can provide the outer surface of the imaging unit 504.

The elongate body 578 can have a proximal section 584, bendable section 586 and distal end section 572 (e.g., distal end 572) arranged in this order from proximal to distal. A joint coupling 588 can couple the proximal section 584 and bendable section 586. The joint coupling 588 can provide a joint to allow for the bending action. A sleeve or other coupling can be used as the joint coupling 588.

The bendable section 586 can have various configurations in order to bendable. Various types of bendable tubes can be adapted to use with the imaging unit 504. Here, the bendable section 586 is configured as shown. The bendable section 586 can include one or more bendable segments 590, which can be configured for cooperating to bend in one or two directions in a plane. Various materials can be used to allow for the bending action and bending between the bendable segments 590. The bendable segments 590 can allow for a larger outside bending radius 592 and a smaller inner bending radius 595, where the smaller inner bending radius 595 is on the side of the bending direction that is opposite of the larger outside bending radius 592. The smaller inner bending radius 595 can range from 5 mm to 20 mm, from 6 mm to 15 mm, or from 7 mm to 10 mm.

In one embodiment, a shape memory member 594 can be included in the bendable section 586. In some instances, a plurality of shape memory members 594 can be used. The shape memory members 594 can be made of a shape memory allow, such as nitinol. When relaxed, the shape memory member 594 is straight. When the articulating force cables 524 is tensioned or pulled toward the base unit 502, the shape memory member 594 can bend to allow for the bendable section 586 to bend as desired. When tension or force is released from the articulating force cables 524, the shape memory member 594 can straighten the bendable section 586.

Referring back to the mechanical system 510, the geared spindle 526 can be operably coupled with drive gear 530. The operably coupling can be by gear teeth 532 of the geared spindle 526 interlacing with gear teeth 534 of the drive gear 530. Accordingly, when the mechanical activating member 514 is activated, the drive gear 530 rotates in a first rotational direction so as to rotate the geared spindle 526 to reel in and wind the force cable 522 into spooled force cable 528. When the mechanical deactivating member 515 is activated, the drive gear 520 releases or rotates in a second rotational direction that is opposite of the first rotational direction so as to release or unwind the force cable 522 from the spooled force cable 528. The mechanical system 510 can include appropriate mechanical components in order to allow for these operations to be performed. For example, the mechanical activating member 514 and mechanical deactivating member 515 can both be operably coupled to a transfer component 536 that transfers the mechanical operations to the drive gear 530. Mechanical components and configuration for the mechanical system 510 can be designed in order to implement the functionality described herein.

In one embodiment, the drive gear 530 can be biased so that it rotates preferentially in one direction when unopposed by the gear spindle 536, mechanical activating member 514, and mechanical deactivating member 515. That is, the drive gear 530 can have a bias by having a bias system 540 operably coupled thereto. The bias system can include a bias element 542 (e.g., spring) coupled to the drive gear 530 in a manner that causes the drive gear 530 to preferentially rotate in one direction or the other. The bias element 542 can be coupled to an anchor 540 that is anchored to the main housing or some other intermediate component. Having one end anchored to the anchor 540 and the other end coupled to the drive gear 530 can apply a constant bias to the drive gear 530. The bias element 542 can cause the force cable 522 to be under tension or under slack depending on the design and orientation. In some instances it can be preferential for the force cable 522 to remain under tension, and in other instances it can be preferential for the force cable 522 to be slack until activated.

In one embodiment, the bias element 542 applies a bias to the drive gear 530 in a manner that applies tension to the force cable 522 through the spindle gear 526. As such, the bias pulls the force cable 522 so as to bend the distal end 572 of the imaging unit 504 if left without other acting forces or restraint. However, the mechanical activating member 514 acts on the drive gear 530 to hold it so that it does not rotate and thereby does not impart additional bias to the force cable 522. As desired, the mechanical activating member 514 can be activated in order to release the drive gear 530 that is biased by the bias element 542, which cases the drive gear 530 to rotate the spindle gear 526 in order to apply additional force or tension to the force cable 522. This function can take the bendable section 586 of the imaging unit 504 from straight to becoming bent or to a further bent position as shown in FIG. 4. The mechanical deactivating member 515 can then be activated to release the tension or force from the force cable 522 so as to allow the bendable section 586 to become straightened.

In one embodiment, the bias element 542 applies a bias to the drive gear 530 in a manner that releases tension to the force cable 522 through the spindle gear 526. As rest, there is no bias to pull the force cable 522 to bend the distal end 572 of the imaging unit 504 if left without other acting forces or restraint, and thereby the bendable section 586 is straight at rest. However, the mechanical activating member 514 can be activated to act on the drive gear 530 so that it rotates, and thereby imparts a bias or tension to the force cable 522. As desired, the mechanical activating member 514 can be activated in order to apply more force or tension to the force cable 522. The applied force to the force cable 522 can take the bendable section 586 of the disposable unit from straight to becoming bent or to a further bent position. The mechanical deactivating member 515 can then be activated to release the tension or force from the force cable 522 so as to allow the bendable section 586 to become straightened.

In one embodiment, the connection between the imaging unit and the base unit can be via magnetic interaction. The connection can include magnets on one side, either the base unit or imaging unit, and either oppositely poled magnets or magnetically responsive materials on the other unit. The connection members described herein can be the magnet and/or magnetically responsive material so long as there is a magnetic field that facilitates the connection between the base unit and imaging unit. In one example, the base unit can have stronger magnets and the imaging unit can have weaker magnets that are less expensive. The magnetic field can also be turned on or turned off when one or more electromagnets are used. These electromagnets can be on the base unit and/or the imaging unit. For example, the magnetic field can be turned on or off during coupling or decoupling of the base unit and imaging unit by turning on or turning off the electrical current to the electro magnet. In one example, the electromagnet can include an electromagnetic coil that can be selectively turned on to provide a magnetic field, and turned off to remove the magnetic field.

In one embodiment, an imaging system can include a base unit and one or more imaging units. The imaging unit can include the elongate member with one or more inflatable members. The inflatable members can be attached and detached from the distal end of the elongate member with or without a fastener. In one aspect, the inflatable members can include a fastener or adhesive rim to couple fit over the distal end and be coupled thereto. The base unit can include: a housing; a system control module located in the housing; a pneumatic control system; a main interface on the housing, the main interface includes a plurality of main electrical connectors operably coupled with the system control module and includes one or more main mechanical connectors; a mechanical system located in the housing and mechanically coupled with the one or more main mechanical connectors; and a mechanical activating member mechanically coupled with the mechanical system. The one or more imaging units can each be configured to be removably coupled to the base unit. Each imaging unit can include: an elongate support member having a proximal end, a proximal section, a bendable section, and a distal end; an imaging interface on the proximal end of the elongate support member, the imaging interface includes a plurality of imaging electrical connectors that correspond and connect with the main electrical connectors of the main interface and includes one or more imaging mechanical connectors that correspond and connect with main mechanical connectors of the main interface; one or more mechanical actuators each having a proximal end mechanically coupled with the one or more imaging mechanical connectors and extending from the imaging interface along the elongate support member to a distal region of the bendable section with is coupled to a distal end of the mechanical actuator; and a plurality of electrically conductive elements electronically coupled with the imaging electrical connectors and disposed within the elongate support member and extending from the imaging interface to an imaging module located in the distal end of the elongate support member. The inflatable member can be included on the distal end of the imaging unit or couplable thereto.

In one embodiment, the main mechanical connectors and imaging mechanical connectors and pneumatic connectors are configured as force transfer connectors when coupled. The force transfer connectors are configured to transfer mechanical forces from the mechanical system of the base unit to the one or more mechanical actuators in order to bend the bendable section of the elongate support member.

In one embodiment, the housing includes a power source that is operably coupled with one or more of the plurality of main electrical connectors, which are configured to transfer electrical power from the base unit to the imaging unit. The force transfer connectors can be adhesive or magnetic. When magnetic, the force transfer connectors include one or more of a permanent magnet, a ferromagnetic member, or an electromagnet. The power source can power the pneumatic system.

In one embodiment, the base unit includes one or more main engagement members associated with the main interface and the imaging unit includes one or more imaging engagement members that are configured to be coupled with the one or more main engagement members when the base unit is coupled to the imaging unit.

In one example, the shaft can be a catheter. In another example, the shaft can be short and screwdriver length. For example, the shaft can have a length of from 0.5 cm to 100 cm, from 0.75 cm to 50 cm, from 1 cm to 15 cm, from 2 cm to 10 cm, or about 5 cm. The coupling interface can include mechanical coupling members and electronic coupling members and pneumatic coupling members for each of the shaft side and handle side.

The proximal end of the shaft can have a coupling member that is configured to couple with a corresponding coupling member on a handle or other base medical device. The handle can have any shape, and may be rounded or ergonomic. The handle or base unit can have a computing system that can be used to perform imaging methods with the imaging device. The computing system can be configured to perform image acquisition, storage, and/or processing.

The interface between the optical elements (e.g., lens) and the imaging module can be direct. The interface between the optical elements and the imaging module can be through one or more optical members, such as one or more optical lens, or other optical elements commonly associated with imaging modules, which is shown in the figures. The lens can be located in lens housing that is between the sheath and the inflatable member. The lens can be set in a distance from the imaging module in order to provide a clear image. The distance can be predetermined. The distance can be adjusted by an adjustment mechanism. The adjustment mechanism can be adjusted on the fly, such as a ring dial that when rotated clockwise moves the lens further from the imaging module and closer to the optical fibers and when rotated counter clockwise moves the lens moves closer to the imaging module and further from the optical fibers, or vice versa. The lens can be rigidly sent in a predetermined optical coupling arrangement to provide a clear image to the imaging module. The distance can be predetermined and optimized and then set.

The invention permits the use of but does not require a relatively large sensor. The light sensor can be dimensioned from 3 to 5 mm as shown in the figures. The imaging device can include a combination of shell light elements arranged in an annular manner around core. A sleeve can encase the lens and sensor, and may also encase the shaft. Because all of the shaft assembly beyond the lens is flexible, the distal end can be moved, bent, or guided as desired. The distal end maneuverability can be determined based on a function of the eventual need, and therefore is decoupled from the fixed dimensions of the sensor and the light source.

The imaging device can include a reduced size, but it can only be so small until there is lost resolution. The size can range from about 0.7 and 0.3 mm for smaller embodiments; however, the size can be as large as desired. The size should be sufficient so that it isn't expensive or fragile. The durability should resist breaking during use. The imaging device can also be configured to be a single use or disposable. In one example, the distal end of the imaging device can be sized small enough to see into spaces between tissues, such as from about ¼ mm to 1 mm. In another example, the diameter can be up to 2 mm. The inflatable member or balloon can be configured to expand up to any desirable and/or reasonable size. The elasticity of the material selected can determine the amount of inflation and thereby the distance of the material from the lens.

The imaging device is configured such that light enters through the inflatable member and to the distal end of the optical elements, and then travels through the optical elements to bring light into the sensor of the imaging module. In one option, the light passes through a lens between the optical elements and sensor in order to focus the light on the sensor. The shaft of the imaging device can be flexibly stiff, with some resilience, but with the capability of significant bending outwardly. The device can also be capable of bending longitudinally as allowed by the optical elements. The inflatable member can be a bladder balloon that is prepared into an inflatable shape, which can be figured to be spherical, rectangular, or any shape.

In one embodiment, the inflatable member can be of a non-elastic or non-stretchable membrane. The inflatable member can be a polymer that is clear that is preshaped into an inflatable member that inflates from a malleable and deflated condition to a partially or fully inflated condition that fills the inflatable member to the preshaped shape. For example, the inflatable member can be a preshaped clear or colored bag or bladder such that inflation is to a predetermined volume. An embodiment may not include an expandable region in a bladder.

In one embodiment, the inflatable member is elastic or stretchable and capable of expanding similar to a balloon. The inflatable member can be prepared from any clear transparent balloon material. Also, colored materials can be used, where the image can be corrected or filtered as desired. The image can be filtered before and/or after being digitized.

In one embodiment, the inflatable member can include a membrane that can be permeable to one or more substances. The membrane can be permeable to the fluid so that constant pressure or flow can be used to maintain inflation or be modulated for varying inflation and maleableness.

In one embodiment, the membrane can be impermeable to one or more substances. The membrane can be inflated as a bladder without stretching or a balloon with stretching.

In one embodiment, the camera receives an image through the wall of the inflatable member. This allows for the camera to receive an image through a transparent bag, bladder, or balloon. The camera can take images through the inflatable member before inflation as well as during or after inflation. Endoscopes can utilize the inflatable member to create space for imaging in fluid or small spaces where there is debris, and murky substances, or the like. The endoscope can inflate the inflatable member to create a void space between the lens and member where the void space is filled with a clear or transparent fluid like water, saline, or clear gas like oxygen, nitrogen or air. The membrane can be configured as a plastic bag that is transparent.

The present invention can include a camera with an inflatable member that is configured for the camera to image through the inflatable member and the inflatable member to inflate to provide a void space for imaging. This can allow imaging in a small cavity or tissue, like a vertebral space or any very small space, in extreme cases, blood vessels. In order to have good imaging, no matter how good the lens or camera is, there is a need for a clear space in front of the lens, and the member inflated, can provide that space.

In order to get good images, it can be beneficial to not contact the imaging area or tissue being imaged with the lens. There is a need for focus—there's no infinitive short focus and even though the full focus now can go down to 3 millimeters, there's still a need to create space or have space between the lens and the imaging area. Also, often there is a medium that is around the imaging area that can include body fluids, blood components, tissues, and debris and other environmental particles that are difficult to image through. Accordingly, the inflatable member can be inflated and pressed against the tissue in order to provide a clear void space between the lens and imaging area. This provides a clear void in front of the camera for enhanced imaging.

Previously, glass domes have been used over lenses. However, these are subject to cracking and breaking, which can leave glass shards that are unfavorable for a living environment or subject. Also, the glass domes are not adjustable and have a constant volume and constant distance between the lens and glass dome. Additionally, sometimes the dome is just too big to operate in a tight space. On the other hand, an inflatable member can be inflated to create an adjustable void space with an adjustable volume and adjustable distance between the lens and member, and thereby an adjustable distance between the lens and imaging area. The inflation can also cause the inflatable member to move the imaging area, such as by pushing a tissue away from the lens.

In one aspect, the inflatable member can be uninflated or collapsed, such as when deploying the camera in a body of a subject. The camera can still image through the collapsed inflatable member (e.g., balloon) on the endoscope for maneuvering and navigation. Accordingly, when the inflatable member balloon collapses down onto the lens, an operator can still see through the inflatable member balloon in order to navigate and perform endoscopic functions in the deflated position and then selectively inflate the inflatable member balloon to change the distance between the inflatable member balloon and lens. The inflatable member balloon can be placed on or adjacent to a tissue or other imaging area for image acquisition.

In one embodiment, the inflatable member can be a membrane, bag, balloon, or bladder or fluid used for inflation thereof can change the refraction index. The camera or imaging module can be configured for correction so that any change in refraction index can be suitable. The clarity of the image can still be obtained for the imaging area.

In one embodiment, the inflatable member can be a membrane that can be permeable to the fluid used for inflation. The device can infuse fluid into the space between the inflatable member and the lens to help clarify the imaging area for enhanced image acquisition. In one embodiment, a port can be adjacent to the inflatable member so that an infusing fluid, such as saline, can wash or cleanse the space adjacent to the imaging area, such as the space between the membrane and the imaging area (e.g., tissue).

In one embodiment, the camera and/or imaging module can be configured to modify the wavelengths of the acquired image for improving or merely modifying an image. The process of modifying wavelength(s) of transmitted light can be used to determine quality/condition/type of tissue being imaged through the membrane. As such, the device, camera, camera sensor, or camera module can incorporate differential sensing elements tuned to wavelengths emitted see previously filed provisional by Ouyang et al: USPTO 61/349514, which is incorporated herein by specific reference.

The optical element can be standard optical elements, such as prisms, optical wedges, lenses, or filters. The filters that can be used in some embodiments may be patentable for this type of micro image sensor. Well known filters include the Bayer filter (e.g., Line 1: G, R, G, R; Line 2: B, G, B, G; repeat) and Diagonal Bayer filters (e.g., Line 1: G, R, G, B; Line 2: B, G, R, G, Line 3: G, B, G, R, Line 4: R G B G; repeat). The filter may be RG/GR pattern using red and green filters to allow sensor to emphasize red and yellow tone: Line 1: R, G, R, G; Line 2: G, R, G, R; repeat). The filter may be RG/BR pattern to allow sensor to emphasize red component which is dominant in surgical tissue images: Line 1: R, G, R, G; Line 2: B, R, B, R; Line 3; repeat). As such, red and pink colors are more prevalent, and the filter has been designed to increase sensitivity to red and pink colors. As such, lower light intensity can provide higher quality images by the filter. In the filters, the letters are as follows: R—Red, G—Green, B—Blue, C—Cyan, M—Magenta, Ye—Yellow, and W—Grey Level (no filter). The filter may be: Line 1: Y, M, Y, M; Line 2: C, Y, C, Y; repeat). The filter may be: Line 1: M, Y, M, Y; Line 2: C, M, C, M; repeat.

In one embodiment, an imaging device can include: an imaging module; a lens associated with the imaging module; and an optically transparent inflatable member surrounding the lens. The imaging device can include an inflation tube fluidly coupled with the inflatable member. The distal end of the imaging device can include an inflation port fluidly coupling the inflation tube and inflatable member. The imaging device can include a fastener that fastens the inflatable member to a housing of the imaging module. In one aspect, the inflatable member is on a distal end of a shaft.

In one embodiment, the imaging device can include a flushing tube fluidly coupled to a flushing port that is directed outward from a housing of the imaging module.

In one embodiment, the imaging device is an endoscope.

In one embodiment, the inflation tube and/or flushing tube are coupled to a fluid source, such as the pneumatic system, that can pump fluid therethrough.

In one aspect, the inflatable member is an inflatable membrane. In one aspect, the inflatable member is a balloon. In one aspect, the inflatable member is a bladder. In one aspect, the inflatable member is a bag. In one aspect, the inflatable member is prepared from a polymer that is transparent. In one aspect, the inflatable member is prepared from an elastomer that is transparent. In one aspect, the inflatable member is prepared from a latex that is transparent. In one aspect, the inflatable member is prepared from polyurethane, nylon, polyethylene, polypropylene, or the like. In one aspect, the inflatable member is stretchable or elastic.

In one aspect, the inflatable member is not stretchable or elastic. In one aspect, the inflatable member is a bag or bladder with a defined volume and/or shape.

In one embodiment, an imaging method can include providing an imaging device with an distal end inflatable member. The imaging device can be used for imaging an imaging area through the inflatable member. The imaging can be while the inflatable member is deflated or inflated. The method can include inflating the inflatable member. The method can include deploying the device with the inflatable member collapsed. The method can include deploying the device with the inflatable member at least partially inflated. The method can include selectively inflating the inflatable member so that the inflatable member is a desired distance from the lens. The method can include deflating the inflated inflatable member. The method can include pressing the inflatable member against the imaging area. The method can include infusing a space adjacent to the imaging area with an infusion solution around the inflatable member.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

1. An imaging device;
an imaging module;
a lens associated with the imaging module; and
an optically transmissive inflatable member surrounding the lens.

2. The imaging device of claim 1, comprising an inflation tube fluidly coupled with the inflatable member.

3. The imaging device of claim 2, comprising an inflation port fluidly coupling the inflation tube and inflatable member.

4. The imaging device of claim 1, comprising a fastener that fastens the inflatable member to a housing of the imaging module.

5. The imaging device of claim 1, comprising a flushing tube fluidly coupled to a flushing port that is directed outward from a housing of the imaging module.

6. The imaging device of claim 5, wherein the inflation tube and/or flushing tube are coupled to a pneumatic system that can pump fluid therethrough.

7. The imaging device of claim 1, wherein the inflatable member is an inflatable membrane.

8. The imaging device of claim 7, wherein the inflatable member is a balloon.

9. The imaging device of claim 1, wherein the inflatable member is a bladder.

10. The imaging device of claim 1, wherein the inflatable member is a bag.

11. The imaging device of claim 1, wherein the inflatable member is prepared from a polymer that is optically transmissive.

12. The imaging device of claim 11, wherein the inflatable member is prepared from an elastomer that is optically transmissive.

13. The imaging device of claim 1, wherein the inflatable member is prepared from a latex that is optically transmissive.

14. The imaging device of claim 11, wherein the inflatable member is prepared from polyurethane, nylon, polyethylene, polypropylene, or the like.

15. The imaging device of claim 1, wherein the inflatable member is on a distal end of a shaft having the imaging module at the distal end.

16. An imaging method comprising:
providing an imaging device having a housing containing an imaging unit with a lens and an optically transmissive inflatable member coupled with the housing with the lens contained within the inflatable member; and
imaging an imaging area through the inflatable member.

17. The method of claim 16, comprising inflating the inflatable member.

18. The method of claim 16, comprising deploying the device to the imaging area to be imaged with the inflatable member collapsed.

19. The method of claim 16, comprising selectively inflating the inflatable member so that the inflatable member is a desired distance from the lens.

20. The method of claim 19, comprising deflating the inflated inflatable member.

21. The method of claim 19, comprising pressing the inflatable member against the imaging area.

22. The method of claim 16, comprising infusing a space adjacent to the imaging area with an infusion solution around the inflatable member.
